(19) United States
(12) Patent Application Publication
(10) Pub. No.: US 2002/0193804 A1
(43) Pub. Date: Dec. 19, 2002

(54) Instrument for inserting tablets into an eye

(76) Inventor: Stephen Paul Tickle, Hertfordshire (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/168,172

(22) PCT Filed: Oct. 19, 2001
(86) PCT No.: PCT/GB01/04663

(30) Foreign Application Priority Data
Oct. 19, 2000 (GB) 0025611.5

(51) Int. Cl. A61F 9/00

(52) U.S. Cl. 606/107

(57) ABSTRACT

A tablet inserter comprises a body portion (12), a plunger (16, 66) which is longitudinally displaceable in the body portion, a dispensing channel (24) which is accessed via a slot (22) for receiving tablets and which is traversed by the plunger, and a cannula (26) at the forward end of the dispensing channel. Forwardly of the access slot (22) there is provided a fluid restrictor comprising an annular collar (42) housed within a cap (40) and held in place by a plug (38). The collar (42) is of resilient plastics material with the aperture therethrough being a sealing fit with the push rod (66).

### FIELD OF THE INVENTION

This invention relates to instruments for the insertion of small tablets into the eye, and in particular into a fluid-filled chamber of the eye.

### BACKGROUND TO THE INVENTION

For the treatment of certain eye conditions there is a need to be able to insert small tablets through an incision in the wall of the eye, without otherwise harming the eye. This is a new procedure and the tablet inserters of the present invention have been developed to meet this need. Instruments in the form of injectors for inserting intraocular lenses into the eye are well-known. However, somewhat different considerations apply when creating an instrument appropriate for the insertion of very small tablets into the fluid of the eye, as compared with the insertion of intraocular lenses through a small incision, as in the removal of cataracts. For example, it is necessary that the fluid in the eye is not able to escape or be withdrawn when the insertion is performed.

### SUMMARY OF THE INVENTION

It is an object of the present invention to provide an instrument suitable for use as a tablet inserter for inserting tablets through an incision in the eye. The instrument is preferably designed and made of materials such that it is reusable, although the instrument could be made in disposable form. Reference herein to “tablets” is to be understood as including both solid items which will gradually dissolve and also capsules which can be filled with a treatment drug and which are designed to dissolve, thereby releasing the contents.

The tablets which are to be inserted into the eye by the instrument of the present invention are preferably cylindrical in shape, in order to enable them more easily to be discharged through a cannula tube of the instrument. Such tablets would typically have a length of about 1.0 mm and a diameter of about 0.38 mm.

In accordance with the invention there is provided an instrument for inserting tablets into a fluid-filled chamber of an eye, comprising a body portion, a plunger which is longitudinally displaceable in the body portion, a dispensing channel at the forward end of the body portion which is arranged to receive tablets to be dispensed and which is traversed by the forward end of the plunger, a cannula at the forward end of the dispensing channel, and restrictor means to prevent the passage of fluid from the eye back through the dispensing channel.

Preferably, the restrictor means comprises a disc of resilient material having an aperture therethrough which is traversed by the plunger upon the dispensation of a tablet. Preferably, the disc aperture is arranged to be enlarged by the passage of the plunger therethrough to provide a sealing fit and becomes of lesser size upon withdrawal of the plunger therefrom.

In a preferred embodiment, the aperture has an inwardly convex circumferential surface when the plunger is absent therefrom, and the disc has a concave circumferential surface in that state.

Preferably, the aperture through the disc has a diameter of the order of 0.36 mm at its outer ends and a diameter at the lengthwise centre of the aperture of the order of 0.15 mm when the plunger is absent.

In this case, the radial depth of the circumferential concavity in the disc is of the order of 0.2 mm at its maximum.

Preferably, the disc is housed within a cap which encircles the dispensing channel and has a passage therethrough forwardly of the disc which forms part of the dispensing channel and through which the plunger is arranged to pass.

Preferably, the plunger is arranged to be held fast in either of two end positions by means of a bayonet fastening.

### BRIEF DESCRIPTION OF THE DRAWINGS

In order that the invention may be more fully understood, one presently preferred embodiment of tablet inserter in accordance with the invention will now be described by way of example and with reference to the accompanying drawings. In the drawings:

FIG. 1 is an external view of the tablet inserter with the plunger depressed;

FIG. 2 is a side view of the inserter of FIG. 1, with the plunger retracted;

FIG. 3 corresponds to FIG. 1, but showing the inserter on a larger scale and partially in section;

FIG. 4 corresponds to FIG. 2, but showing the inserter on a larger scale and partially in section;

FIG. 5 is a side view of the main body of the inserter;

FIG. 6 is a view of the main body of FIG. 5, rotated through 90°;

FIG. 7 is the sectional view taken along the line VII-VII in FIG. 6;

FIG. 8 is the front end view of a restriction collar to be fitted in the main body of the inserter;

FIG. 9 is the side view of the restriction collar of FIG. 8;

FIG. 10 is a side view of a restriction cap within which the restriction collar is fitted;

FIG. 11 is a plan view of the plunger which is fitted within the main body of the inserter;

FIG. 12 is a side view of the plunger of FIG. 11;

FIG. 13 shows the push rod which is displaceable by the plunger;

FIG. 14 shows the cannula tube; and

FIG. 15 is a side view of a plug which is fitted within the nose of the main body of the injector.

### DESCRIPTION OF THE PREFERRED EMBODIMENT

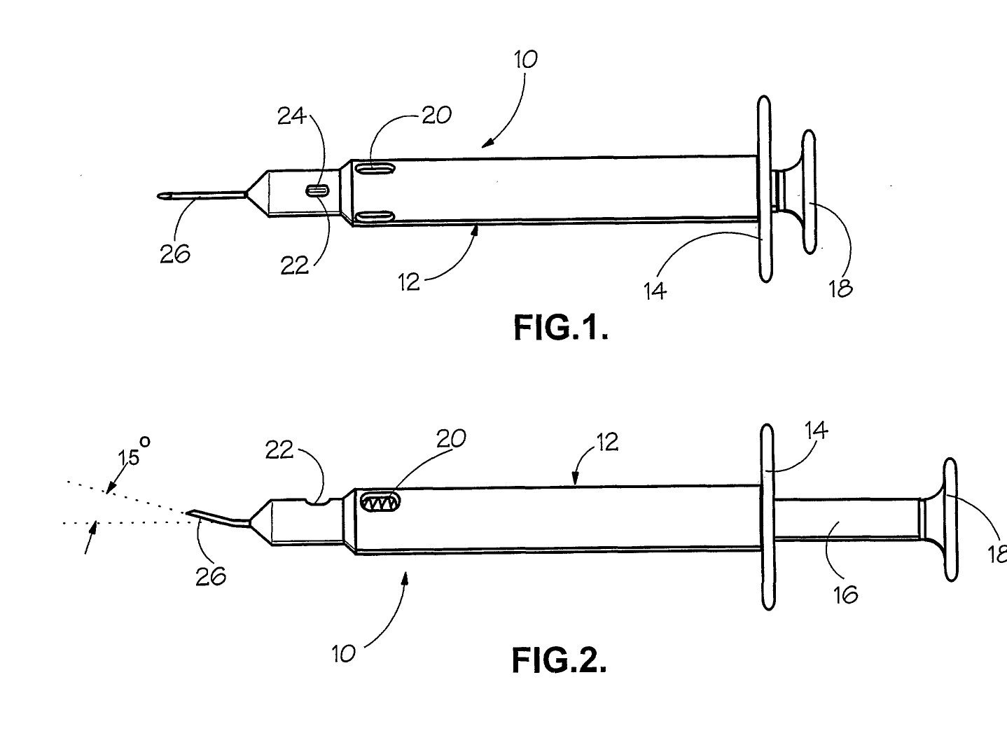

FIGS. 1 and 2 show the tablet inserter 10 as comprising a main body portion 12, a flange 14 and a plunger 16 which has an end pad 18. Towards the front end of the main body portion 12 are three equispaced viewing apertures 20. In the forward portion of the main body, which is of slightly reduced diameter, there is provided an access slot 22, at the bottom of which is a channel 24 for receiving tablets inserted into the access slot. Projecting from the forward end of the main body portion 12 is a cannula tube 26 which, as shown in FIG. 2, is angled at 15° to the longitudinal axis of the instrument over a part of its length.

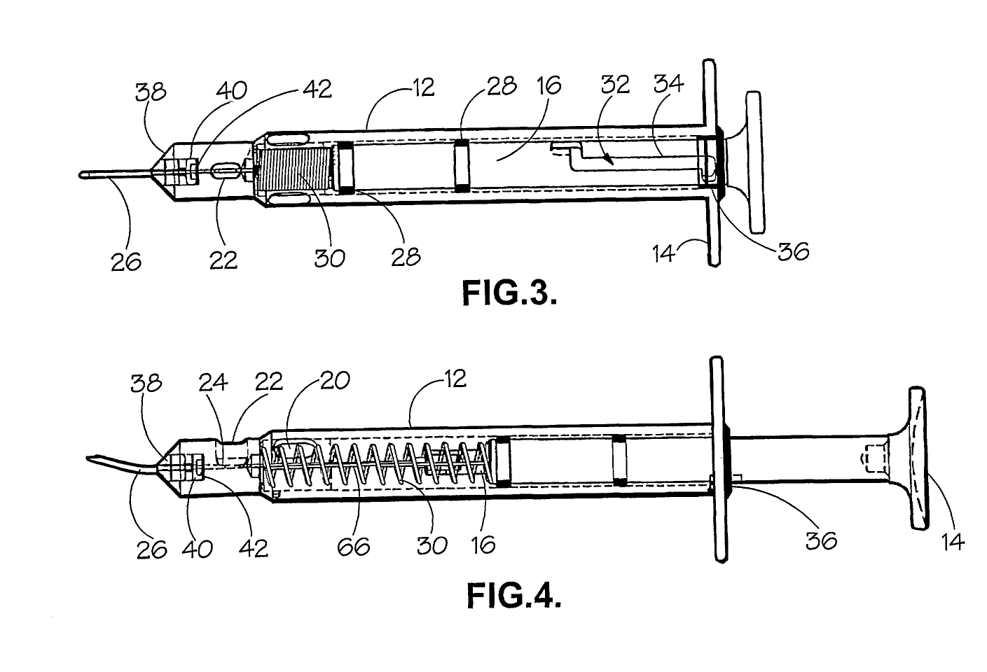

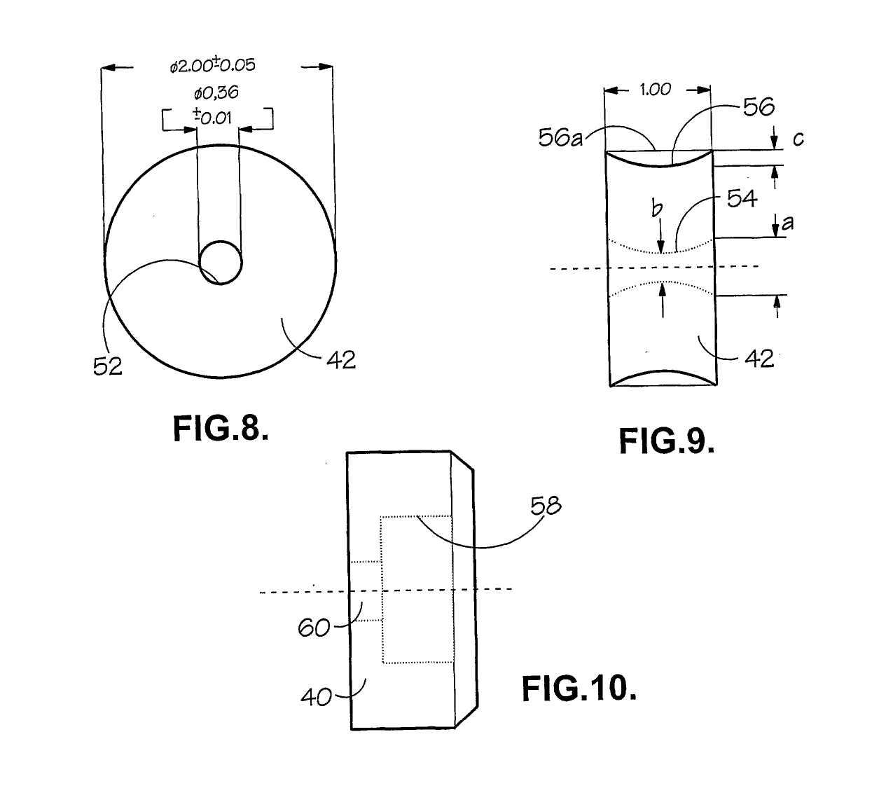

FIGS. 3 and 4 show more details of the inserter. Around the plunger 16 are a pair of friction pads 28 and the plunger is biased towards its retracted position by a helical spring 30. As shown in FIG. 3, the plunger 16 incorporates a bayonet fastening, indicated generally at 32, by means of which it can be “locked” in either of two positions, namely a retracted position and a depressed position. The bayonet fastening 32 comprises a surface slot 34 in the plunger and a stop pin 36 which projects radially inwardly at the rearward end of the main body portion 12 and which is engageable with the bayonet slot 34. Thus, the plunger 16 can be locked in its fully depressed position and also in its fully retracted position. Also shown in FIGS. 3 and 4 are a front end plug 38, which is shown in more detail in FIG. 15, a restriction cap 40, which is shown in more detail in FIG. 10, and a restriction collar 42, which is shown in more detail in FIGS. 8 and 9.

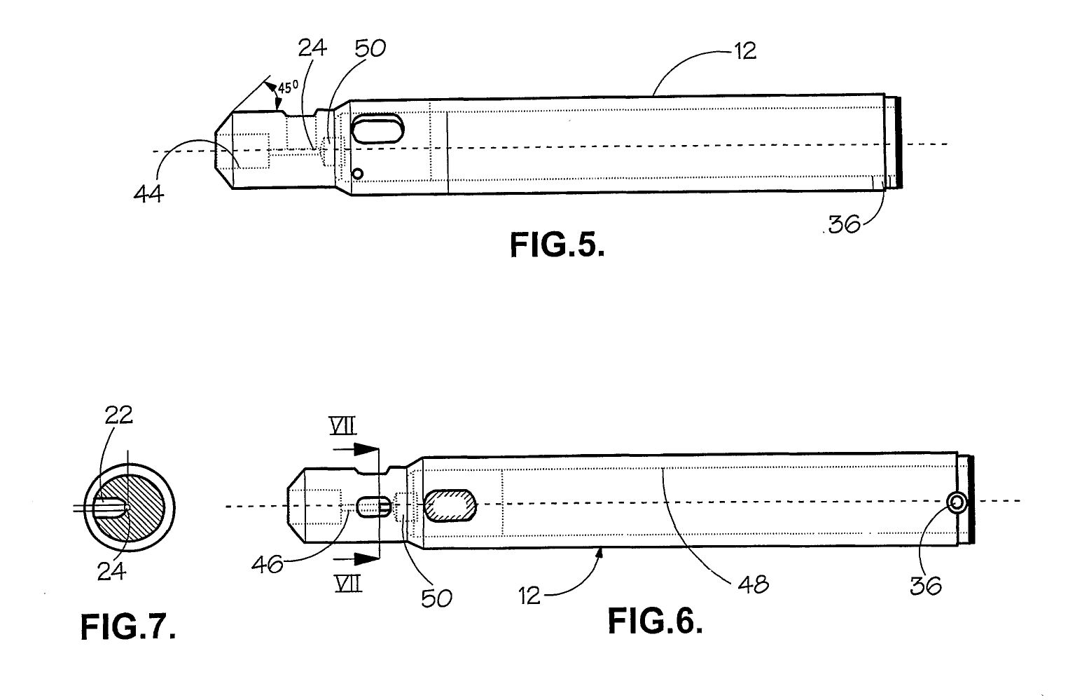

FIGS. 5 and 6 show more details of the main body portion 12 of the instrument. In particular, these Figures show that the forward end of the main body portion is provided with a cylindrical recess 44 into which are fitted the end plug 38, restriction cap 40 and restriction collar 42. From the rearward end of this recess 44 extends a short bore 46 which is a forward extension of the channel 24 into which the tablets are placed. The rearward portion of the main body is provided over its full length with a cylindrical recess 48 which receives the displaceable plunger. At the forward end of the recess 48 there is provided a counterbore 50 into which the forward end of the plunger 16 is received when the plunger is fully depressed. This counterbore 50 provides a seat for the plunger in this setting.

Referring now to FIGS. 8 and 9, these show the restriction collar 42 which takes the form of a cylindrical collar of plastics material, for example produced from a sheet of silicone material such as is supplied under the trade mark “Pharmelast”. The collar has a nominal diameter of 2.0 mm and a length of 1.0 mm. Through the centre of the collar is an aperture 52 which, as shown in FIG. 9, is of variable size. In its “rest” position the aperture 52 has an inwardly convex circumferential wall 54, as indicated by the broken lines in FIG. 9. The circumferential surface 56 is then concave. However, when a push rod connected to the forward end of the plunger is pushed forwards through the restriction collar 42, the aperture wall 54 has sufficient resilience to be pushed outwards and to create a sealing fit with the plunger. The concavity of the circumferential wall 56 enables this outward movement of the material to be accommodated, by the outer wall being displaced into the position 56a where it is in sealing contact with an encircling cap 40 (FIG. 10). In a preferred embodiment, where the central aperture 52 has a diameter a of 0.36 mm at its outer ends, the diameter b of the aperture at the centre, in the zone of minimum cross-section, is 0.15 mm in the rest position. The depth c of the circumferential concavity is approximately 0.2 mm at its maximum.

FIG. 10 shows the restriction cap 40, which is essentially cylindrical, with a chamfered rear end. The cap 40 is made of titanium alloy. The cap 40 is provided with a rearward recess 58 of cylindrical shape and which is of the same diameter as the external diameter of the restriction collar 42, so that the collar 42 will fit within the recess 58, in the cap 40. The restriction cap 40 is also provided with an aperture 60 from its forward end back to the cylindrical recess 58 to provide for the passage of the push rod therethrough. With a collar having the dimensions given above, the aperture 60 is of the order of 0.5 mm in diameter. It will be appreciated that with the restriction collar in place within the cap there is a good seal against the flow of any fluid past the collar in the rearward direction, due to the sealing fit of the collar around the push rod and the relatively small annular surface at the forward end of the collar 42 which is common to the aperture 60. There is therefore very little exposed surface area on the end face of the collar against which fluid can exert pressure.

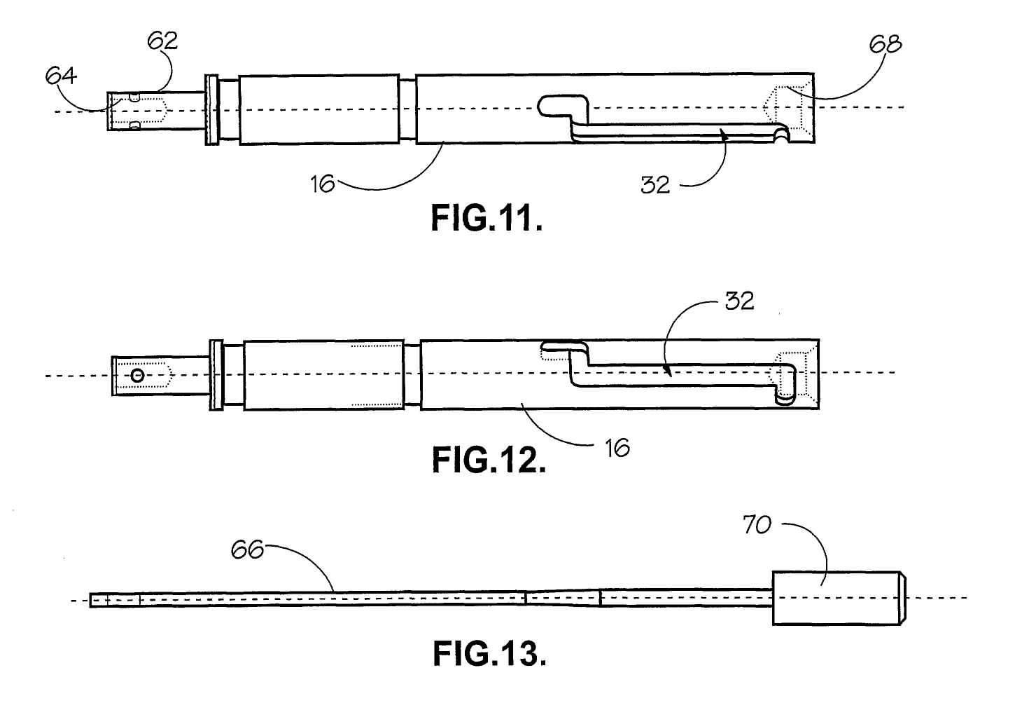

FIGS. 11 and 12 show further details of the plunger 16. The forward end of the plunger is provided with a reduced cross-section portion 62 which is provided with a recess 64 to receive the rear end of a push rod 66 (FIG. 13). There is provided a pin connection between the rearward end of the push rod 66 and the forward end 62 of the plunger. The rearward end of the plunger 16 is provided with a recess 68 which receives the flange 14.

FIG. 13 shows details of the push rod 66. This is made from a plastics material such as PEEK. It has a forward end diameter of 0.38 mm which matches that of the tablets which are to be inserted using the instrument. The rearward end of the push rod has a large diameter cylindrical portion 70 which is connected by a pin to the forward end of the plunger as described above. As can be seen most clearly from FIGS. 3 and 4, the push rod 66 of plastics material is arranged to traverse the channel 24 below slot 22, the restriction collar 42, the restriction cap 40 and the front end plug 38 and to enter into the cannula tube 26 for dispensation of the tablets. In its retracted position, as shown in FIG. 4, the front end of the push rod 66 is positioned just to the rear of the access slot 22.

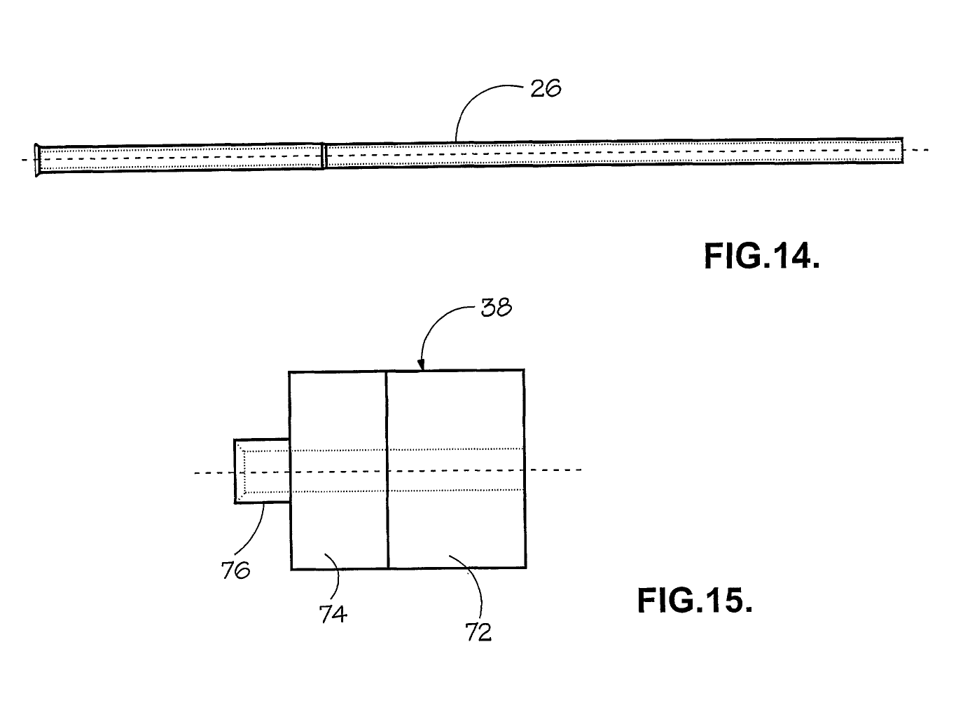

FIG. 14 shows further details of the cannula tube 26. This is made of titanium alloy and can be cut to length prior to fitting to the instrument. As an example, it can be of the order of 15.5 mm long.

FIG. 15 shows the front end plug 38 which holds the restriction cap 40 and collar 42 in place. It again is made of titanium alloy and receives the cannula tube 26 at its forward end. As shown in FIG. 15, it comprises a cylindrical rearward portion 72, a cylindrical central portion 74 of slightly reduced diameter, and a nose portion 76 which has an internally flared end to receive the cannula tube 26.

If the instrument is to be reusable then the component parts, apart from the restriction collar 42 and push rod 66, are preferably of a titanium alloy material. The push rod 66 and restriction collar 42 are of appropriate plastics material. If the instrument is intended to be a disposable instrument, then alternative materials may be utilised.

Although tablets of a diameter of approximately 0.38 mm are dispensable with this instrument, tablets of larger or smaller dimensions may equally well be used, with appropriate changes to the dimensions of the component parts of the instrument.

In use, one or more tablets are inserted through the access slot 22 into the channel 24, with the plunger and push rod in their retracted position, into which they are biased by the spring 30. Then, by unlocking the bayonet connection and depressing the plunger, the push rod is advanced through the channel 24 and forces the tablets forwards through the cannula 26 and into an incision in the eye. By the provision of the bayonet fastening and the use of the restriction cap and collar one minimises the possibility of visco-elastic fluid being sucked back from the interior of the eye.

1. An instrument for inserting tablets into a fluid-filled chamber of an eye, comprising a body portion, a plunger which is longitudinally displaceable in the body portion, a dispensing channel at the forward end of the body portion which is arranged to receive tablets to be dispensed and which is traversed by the forward end of the plunger, a cannula at the forward end of the dispensing channel, and restrictor means to prevent the passage of fluid from the eye back through the dispensing channel.

2. An instrument as claimed in claim 1, in which the restrictor means comprises a disc of resilient material having an aperture therethrough which is traversed by the plunger upon the dispensation of a tablet.

3. An instrument as claimed in claim 2, in which the disc aperture is arranged to be enlarged by the passage of the plunger therethrough to provide a sealing fit and becomes of lesser size upon withdrawal of the plunger therefrom.

4. An instrument as claimed in claim 2 or 3, in which the aperture has an inwardly convex circumferential surface when the plunger is absent therefrom, and the disc has a concave circumferential surface in that state.

5. An instrument as claimed in claim 4, in which the aperture through the disc has a diameter of the order of 0.36 mm at its outer ends and a diameter at the lengthwise centre of the aperture of the order of 0.15 mm when the plunger is absent.

6. An instrument as claimed in claim 4 or 5, in which the radial depth of the circumferential concavity in the disc is of the order of 0.2 mm at its maximum.

7. An instrument as claimed in any of claims 2 to 6, in which the disc comprises a cylindrical collar of plastics material having a diameter of the order of 2 mm and a length of the order of 1 mm.

8. An instrument as claimed in any of claims 2 to 7, in which the disc is housed within a cap which encircles the dispensing channel and has a passage therethrough forwardly of the disc which forms part of the dispensing channel and through which the plunger is arranged to pass.

9. An instrument as claimed in any preceding claim, in which the plunger comprises a push rod having a forward end having a diameter substantially equal to the diameter of the dispensing channel.

10. An instrument as claimed in any preceding claim, in which at the forward end of the body portion there is provided a radial access slot at the bottom of which is the dispensing channel, tablets being arranged to be inserted into the channel via the access slot.

11. An instrument as claimed in any preceding claim, in which the cannula is set at an angle of the order of 15° to the longitudinal axis of the body portion.

12. An instrument as claimed in any preceding claim, in which the plunger is arranged to be held fast in either of two end positions by means of a bayonet fastening.
